(19) United States
(12) Patent Application Publication
(10) Pub. No.: US 2020/0219608 A1
(43) Pub. Date: Jul. 9, 2020

(54) SYSTEM AND METHOD FOR MONITORING AND MANAGING A COGNITIVE LOAD OF A PERSON

(71) Applicant: Intuition Robotics, Ltd., Ramat-gan (IL)

(72) Inventors: Roy AMIR, Mikhmoret (IL); Itai MENDELSOHN, Tel Aviv-Yafo (IL); Dor SKULER, Oranit (IL); Shay ZWEIG, Harel (IL)

(73) Assignee: Intuition Robotics, Ltd., Ramat-Gan (IL)

(21) Appl. No.: 16/737,247

(22) Filed: Jan. 8, 2020

Related U.S. Application Data
(60) Provisional application No. 62/789,744, filed on Jan. 8, 2019.

(51) Int. Cl. G16H 20/70; G16H 50/30; G16H 10/60; G06N 20/00; G06N 5/04; H04L 29/08

(52) U.S. Cl. CPC G16H 20/70; G16H 50/30; G16H 10/60; G16Y 10/60; G06N 5/04; H04L 67/12; G06N 20/00

(57) ABSTRACT

A system and method for monitoring and managing a cognitive load of a person, including: determining, based on an analysis of at least one input data associated with a person, a current cognitive load score of the person; determining, based on the analysis of the at least one input data and the determined current cognitive load score, whether a reduction of the current cognitive load score of the person is desirable; and selecting at least one predetermined plan for execution based on when a reduction of the current cognitive load score of the person is desirable.

### CROSS-REFERENCE TO RELATED APPLICATIONS

This application claims the benefit of U.S. Provisional Application No. 62,789,744 filed on Jan. 8, 2019, the contents of which are hereby incorporated by reference.

### TECHNICAL FIELD

The present disclosure relates generally to measuring cognitive load, and more specifically to a system and method for monitoring and managing cognitive load of a person.

### BACKGROUND

Sophisticated devices configured to provide help to individuals are becoming more commonplace with the increased prevalence of inexpensive computing power, smart devices, and artificial intelligence. These devices include gadgets of the so called “Internet of Things,” or IoT devices, designed to provide advanced “smart” features to everyday appliances and household devices. Some are being introduced within the home or office, and may serve as a form of a digital companion assistant to many. For example, some companies have been testing and introducing helper robots within the retail space as digital greeters and assistants programmed to help patrons navigate a store and find a section or a particular item. Within the home, personal devices are used to provide companionship and assistance to individuals, such as by creating reminders and shopping lists, or playing music either on command, or at predetermined times.

One aspect of providing effective assistance is determining how much focus and cognitive load is desired or required of a user in various scenarios, such as an elder needing to take medication, or a parent seeking assistance when shopping with children, in addition to anticipating changes in required focus. Determining the cognitive load of a person, and when decreasing the load is desirable, is a challenging task to successfully determine.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “certain embodiments” may be used herein to refer to a single embodiment or multiple embodiments of the disclosure.

Certain embodiments disclosed herein include a method for monitoring and managing a cognitive load of a person, including: determining, based on an analysis of at least one input data associated with a person, a current cognitive load score of the person; determining, based on the analysis of the at least one input data and the determined current cognitive load score, whether a reduction of the current cognitive load score of the person is desirable; and, selecting at least one predetermined plan for execution based on when a reduction of the current cognitive load score of the person is desirable.

Certain embodiments disclosed herein also include a non-transitory computer readable medium having stored thereon instructions for causing a processing circuitry to perform a process, the process including: determining, based on an analysis of at least one input data associated with a person, a current cognitive load score of the person; determining, based on the analysis of the at least one input data and the determined current cognitive load score, whether a reduction of the current cognitive load score of the person is desirable; and, selecting at least one predetermined plan for execution based on when a reduction of the current cognitive load score of the person is desirable.

Certain embodiments disclosed herein also include a system for monitoring and managing a cognitive load of a person, including: a processing circuitry; and a memory, the memory containing instructions that, when executed by the processing circuitry, configure the system to: determine, based on an analysis of at least one input data associated with a person, a current cognitive load score of the person; determine, based on the analysis of the at least one input data and the determined current cognitive load score, whether a reduction of the current cognitive load score of the person is desirable; and, select at least one predetermined plan for execution based on when a reduction of the current cognitive load score of the person is desirable.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram of a system utilized for monitoring and managing a cognitive load of a person according to an embodiment.

FIG. 2 is a block diagram of a controller according to an embodiment.

FIG. 3 is a flowchart illustrating a method for monitoring and managing cognitive load of a person according to an embodiment.

FIG. 4 is a flowchart illustrating a method for monitoring and managing interactions based on a cognitive load of a person according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

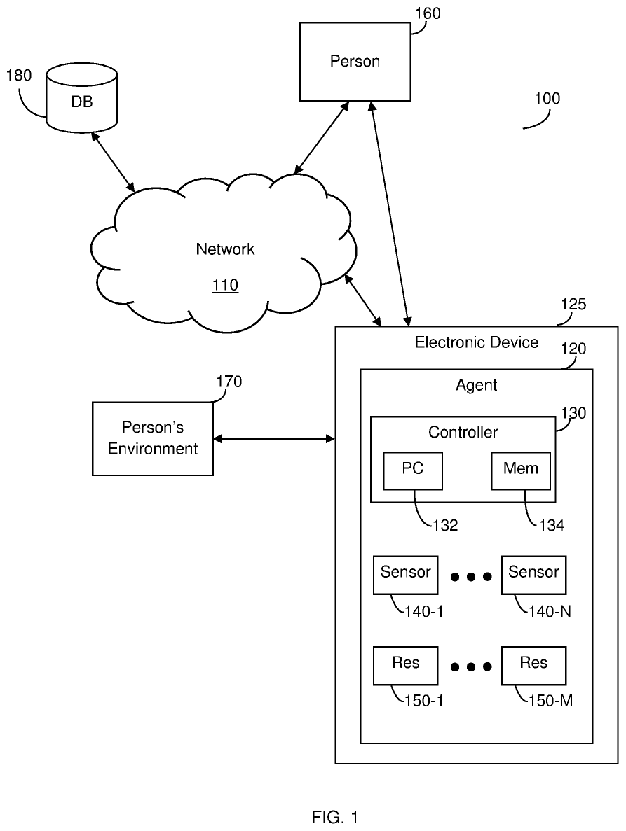

FIG. 1 is an example network diagram of a system 100 according to an embodiment. The system 100 includes an agent 120, an electronic device 125, a person 160 and an environment of the person 170. In some embodiments, the agent 120 is further connected to a network, where the network 110 is used to communicate between different parts of the system 100. The network 110 may be, but is not limited to, a local area network (LAN), a wide area network (WAN), a metro area network (MAN), the Internet, a wireless, cellular or wired network, and the like, and any combination thereof.

The person 160 may access the agent 120 directly, e.g., via a voice command or an input device connected directly to the agent 120, or indirectly through the network 110, e.g., through an application on a mobile phone connected to the internet, where the agent 120 is additionally connected to the internet. Further, the person 160 may access the agent 120 through the electronic device 125 in which the agent resides, as discussed further below. The agent 120 may include hardware, software, a combination thereof, and the like. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions cause a processing circuitry to perform the various processes described herein.

The agent 120 may be connected to, or implemented on, the electronic device 125. The electronic device 125 may be for example and without limitation, a robot, a social robot, a service robot, a smart TV, a smartphone, a wearable device, a vehicle, a computer, smart appliances, and the like.

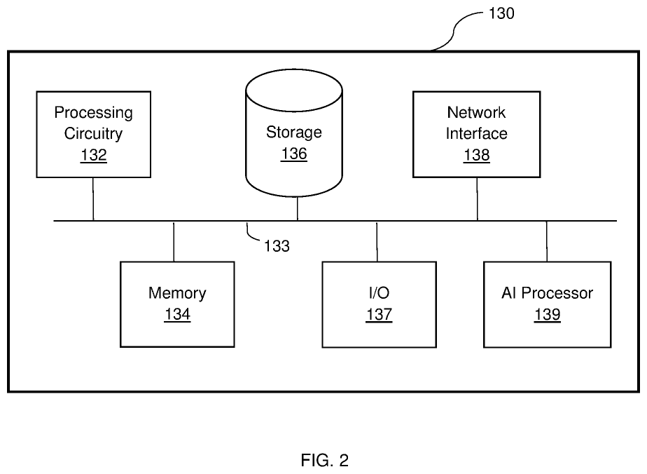

The agent 120 includes a controller 130, explained in more detail below in FIG. 2, having at least a processing circuitry 132 and a memory 134. The agent 120 may further include, or is connected to, one or more sensors 140-1 to 140-N, where N is an integer equal to or greater than 1 (hereinafter referred to as sensor 140 or sensors 140 merely for simplicity) and one or more resources 150-1 to 150-M, where M is an integer equal to or greater than 1 (hereinafter referred to as resource 150 or resources 150 merely for simplicity). The resources 150 may include display units, audio speakers, lighting system, and the like. In an embodiment, the resources 150 may encompass sensors 140 as well.

The sensors 140 may be may include input devices, such as various sensors, detectors, microphones, touch sensors, movement detectors, cameras, and the like. Any of the sensors 140 may be, but are not necessarily, communicatively or otherwise connected to the controller 130 (such connection is not illustrated in FIG. 1 merely for the sake of simplicity and without limitation on the disclosed embodiments). The sensors 140 may be configured to sense signals received from the person 160, the person's environment 170, and the like. The sensors 140 may be positioned on or connected to the electronic device 125.

The agent 120 is configured to use the controller 130, the sensors 140, and the resources 150 in order to monitor and manage a cognitive load of the person 160 as further described herein below. Cognitive load refers to the mental effort being used by the person that is concentrating in one or more tasks such as reading a book, texting, talking on the phone, writing an email, interacting with other people, interacting with the agent 120 or other electronic systems, and so on.

In one embodiment, the system 100 further includes a database 180. The database 180 may be stored within the agent 120 (e.g., within a storage device not shown), or may be separate from the agent 120 and connected thereto via the network 110. The database 180 may store one or more plans to be executed for reducing the person's cognitive load, using the resources 150, based on determination that reduction of the current cognitive load score of the person is desirable, as further discussed herein below.

According to another embodiment, the database 180 may have stored therein historical data associated with the person 160. The historical data may be retrieved from the database 180 and used to determine, for example, the most effective way for using the resources 150 with respect to a specific identified person 160.

FIG. 2 shows a schematic block diagram of a controller 130 of an agent, e.g., the agent 120 of FIG. 1, according to an embodiment. The controller 130 includes a processing circuitry 132 configured to receive data, analyze data, generate outputs, and the like, as further described herein below. The processing circuitry 132 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The controller 130 further includes a memory 134. The memory 134 may contain therein instructions that, when executed by the processing circuitry 132, cause the controller 130 to execute actions as further described herein below. The memory 134 may further store therein information, e.g., data associated with predetermined plans that may be executed by one or more resources, e.g., resources 150 of FIG. 1. As discussed above, the resources 150 include means by which the agent 120, interacts with at least one person, e.g., the person 160, collects data related to the person, and the like. The resources 150 may include, for example, electro-mechanical elements, sensors, detectors, display units, speakers, microphones, touch sensors, light sensors, movement detectors, cameras, and so on as further described herein.

In an embodiment, the controller 130 includes a network interface 138 configured to connect to a network, e.g., the network 110 of FIG. 1. The network interface 138 may include, but is not limited to, a wired interface (e.g., an Ethernet port) or a wireless port (e.g., an 802.11 compliant Wi-Fi card) configured to connect to a network (not shown).

The controller 130 further includes an input/output (I/O) interface 137 configured to control the resources 150 that are connected to the agent 120. In an embodiment, the I/O interface 137 is configured to receive one or more signals captured by sensors 140 of the agent 120 and send them to the processing circuitry 132 for analysis. According to one embodiment, the I/O interface 137 is configured to analyze the signals captured by the sensors 140, detectors, and the like. According to a further embodiment, the I/O interface 137 is configured to send one or more commands to one or more of the resources 150 for executing one or more plans or capabilities of the agent 120.

A plan may include for example, an active action such as generating a notification on a display unit of a social robot (not shown), turn down the volume of music being played by a sound system, stop other interactions that may be determined to be distractive to the person, and the like. The plan may also include a passive action, e.g., by preventing the generation of new interactions that may be initiated by the agent or other electronic systems with the person, when reducing the cognitive load of the person, or maintaining a low cognitive load, is desirable. Reducing the cognitive load of a person may be desirable when, for example, a certain scenario that requires the person's attention presents itself. Examples of such scenarios are further discussed herein below. According to a further embodiment, the components of the controller 130 are connected via a bus 133.

The controller 130 further includes an artificial intelligence (AI) processor 139. The AI processor 139 may be realized as one or more hardware logic components and circuits, including graphics processing units (GPUs), tensor processing units (TPUs), neural processing units, vision processing unit (VPU), reconfigurable field-programmable gate arrays (FPGA), and the like. The AI processor 139 is configured to perform machine learning based on sensory inputs received from the I/O unit 137, which receives input data, such as sensory inputs, from the sensors 140. In an embodiment, the Al processor 139 is further configured to determine, based on one or more machine learning models, the current state of one or more persons, the current cognitive load of the person, and so on. In an embodiment, the Al processor 139 is further configured to select and execute one or more predetermined plans when the cognitive load of the person is relatively high and the scenario requires a reduction of the cognitive load of the person in order to, for example, prevent a human error, perform a delicate operation properly, and so on.

In an embodiment, the controller 130 is configured to receive at least one input data that may be associated with at least one of: the person, the person's environment, an interaction between the person and at least one entity, and the like. The input data may include sensory inputs received from one or more sensors 140, information received from databases, websites, other electronic sources, such as the person's electronic calendar, social media accounts, the Internet, and the like. The input data may indicate, as non-limiting examples, if the person is reading, eating while watching TV, talking with another person in a room, cutting vegetables with a sharp knife, making chocolate, walking in a crowded area, and so on. The entity with which the person may interact may refer to, e.g., other people, the agent 120 of FIG. 1, a smartphone, a service robot, a computer, and the like.

The controller 130 is configured to analyze the input data. The analysis may be performed using, for example and without limitations, one or more computer vision techniques, audio signal processing techniques, machine learning techniques, and the like.

The controller 130 may be configured to determine, based on the analyzed at least one input data, a current cognitive load score of the person. The current cognitive load score is indicative of the mental effort currently being used by the person, e.g., while they are concentrating one or more tasks such as reading a book, texting, talking on the phone, writing an email, interacting with other people, interacting with the agent or other electronic systems, in a crowded area, and the like. Determining the current cognitive load score of the person may be achieved using, for example, at least one machine learning technique.

As a non-limiting example, when the person is talking on the phone and simultaneously writing an email, the cognitive score may be relatively high, indicating the person is currently employing significant mental effort. According to another example, if the person is sitting in a quiet room and listen to music, the cognitive load score may be relatively low.

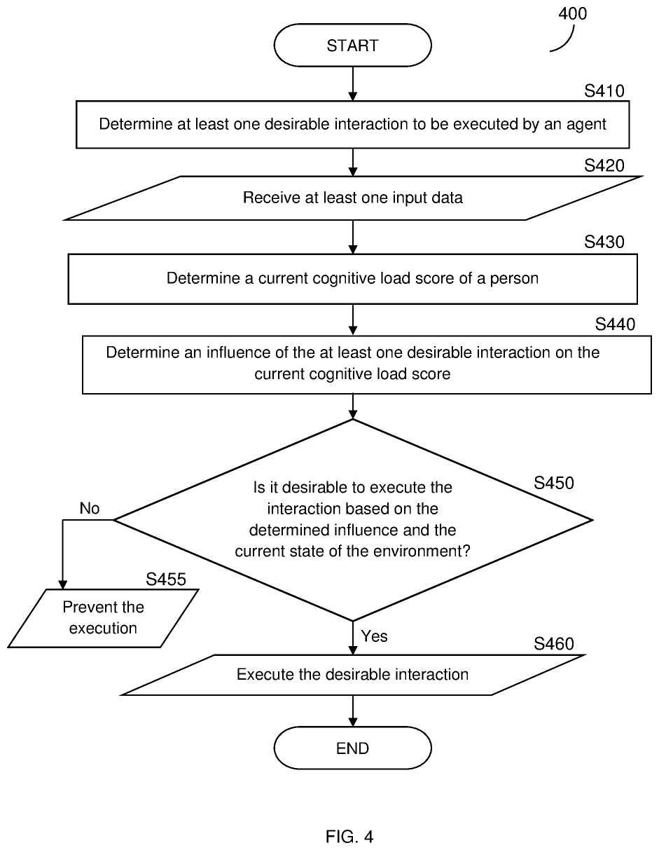

According to another embodiment, the determination of the current cognitive load score of the person may further include analyzing interaction data between the agent and the person. Interaction data refers to information that is associated with a current interaction between the person and the agent, an upcoming interaction between the person and the agent, and the like. That is, in some cases the agent may initiate an interaction with the person, for example to update the person regarding the weather forecast, recommend on a recipe, and so on. This interaction may distract the person's attention and may increase the cognitive load score. Thus, the interaction with the agent may also affect the cognitive load score of the occupant. In an embodiment, the controller 130 may determine that an interaction that is supposed to be executed by the electronic device shall not be executed due to a relatively high current cognitive load score of the person as further discussed in FIG. 4.

The controller 130 may be configured to determine based on the analysis of the at least one input data and the determined cognitive load score whether a reduction of the current cognitive load score of the person is desirable. Determining whether it is desirable or not to reduce the current cognitive load of the person may be achieved using at least one machine learning technique. In an embodiment, the machine learning technique includes implementation of one or more neural networks, recurrent neural networks, decision tree learning, Bayesian networks, clustering, and the like, based on the sensory inputs. As a non-limiting example, a social robot that reminds an elder to take medications every day at 5 μm stops all interactions with the person when it is time to take the medications in order to reduce their cognitive load. In the above example, the input data may be a reminder that was previously determined by the person or learned by an agent of, e.g., a social robot (not shown).

As another non-limiting example, a service robot in a mall may be configured to escort customers and help with their shopping by, for example, physically carrying the bags, and giving recommendations and directions about what and where to shop. When the service robot, i.e., an agent within the robot, identifies that a person has two children that are walking next to him or her in a crowded area, the robot may be configured to reduce distractions for the person by, for example, preventing the generation of shopping recommendations. According to the same example, if the agent senses that the person is currently not attentive, he will direct the person's attention to the children, e.g., via an auditory or visual cue, to make sure they do not become lost.

As another non-limiting example, when a kitchen assistance robot assists a chef in making a dish and reaches a delicate part in the recipe, such as a point where the person needs to watch the exact temperature when making chocolate, it will be desirable to reduce disturbances, set the right music for concentration, refrain from suggesting any unrelated or irrelevant tips, and so on.

The controller 130 may be configured to select at least one predetermined plan for execution based on a determination that a reduction of the current cognitive load score of the person is desirable. The plan may be selected from a plurality of predetermined plans that may be stored in a database, e.g., the database 180. The selected plan may include for example, turning down the volume of a sound system, generating an indication on a display unit of a robot, refraining from interacting with the person, and so on.

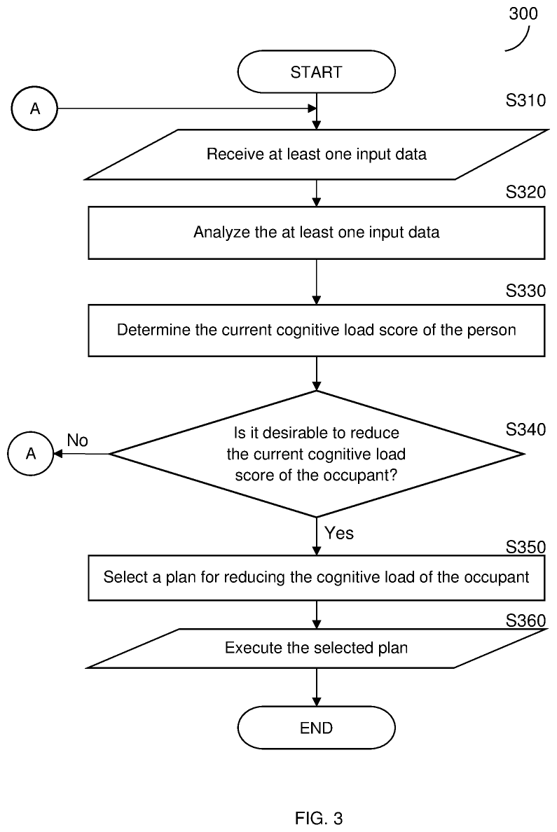

FIG. 3 is an example flowchart 300 illustrating a method for monitoring and managing cognitive load of a person. At S310, at least one input data is received, e.g., by the agent 120 of FIG. 1 that is connected to at least one electronic device. The input data may be associated with, for example, the person directly, the environment of the person, an interaction between the person and at least one entity, and the like, as further described herein above.

At S320, the input data is analyzed. The analysis may be achieved using, for example, at least one computer vision technique or a machine learning technique. In an embodiment, the machine learning technique includes implementation of one or more neural networks, recurrent neural networks, decision tree learning, Bayesian networks, clustering, and the like, based on the sensory inputs.

At S330, a current cognitive load score of the person is determined based on the analysis of the input data. The determination of the current cognitive load score of the person may be achieved using at least one machine learning technique.

At S340, it is determined, based on the analysis of the at least one input data and the determined cognitive load score, whether a reduction of the current cognitive load score of the person is desirable and if so, execution continues with S350; otherwise, execution continues with S310.

At S350, at least one predetermined plan is selected based on determination that reduction of the current cognitive load score of the person is desirable. The determination whether a reduction of the current cognitive load score of the person is desirable may be achieved using at least one machine learning technique.

At S360, the selected plan is executed. The execution of the selected predetermined plan may be made using at least one resource, such as the resources 150 of FIG. 1. The resources 150 may be related to, for example, the electronic device 125 and controlled by the agent 120, i.e., via the controller 130.

FIG. 4 is an example flowchart 400 illustrating a method for monitoring and managing interactions executed by an agent that is communicatively connected to a controller of an electronic device based on a cognitive load of a person according to an embodiment.

At S410, at least one desirable interaction to be executed by an electronic device, e.g., by an agent 120 of the electronic device 125 of FIG. 1, is determined. As an example, a desirable interaction is a proactive communication program. The proactive communication program may be desirable in several scenarios, such as when the person is determined to be not active enough, sad, bored, crying, when the weather changes and a proactive notification may be desirable, and the like.

At S420, at least one input data is received, e.g., by the agent of the electronic device. The input data may be associated with, for example, the person, the environment of the person, an interaction between the person and at least one entity, and so on as further described herein above.

At S430, a current cognitive load score of the person is determined based on the analysis of the input data. The determination of the current cognitive load score of the person may be achieved using at least one machine learning technique.

At S440, an influence of the at least one desirable interaction on the current cognitive load score is determined. The influence may be, reducing the cognitive load score or increasing the cognitive load score. In an embodiment, the desirable interaction may have no influence on the current cognitive load score.

At S450, it is determined whether it is desirable to execute the determined interaction based on the determined influence and the at least one input data, and if so, execution continues with S460; otherwise, execution continues with S455. For example, if the current cognitive load score of the person is 3 out of 5, and the person is taking medications, a desirable interaction that will suggest watching a lecture may increase the cognitive load score to 4 out 5, which may be undesirable as it may be distractive with respect to the activity of taking medication, and should therefore be prevented.

At S455, upon determination that it is not desirable to execute the interaction the execution is prevented.

At S460, upon determination that it is desirable to execute the interaction, the interaction is executed.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; A and B in combination; B and C in combination; A and C in combination; or A, B, and C in combination.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for monitoring and managing a cognitive load of a person, comprising:
determining, based on an analysis of at least one input data associated with a person, a current cognitive load score of the person;
determining, based on the analysis of the at least one input data and the determined current cognitive load score, whether a reduction of the current cognitive load score of the person is desirable; and,
selecting at least one predetermined plan for execution based on when a reduction of the current cognitive load score of the person is desirable.

2. The method of claim 1, wherein the analysis is of the at least one input data associated with the person is performed using at least one of: a computer vision technique, an audio signal processing technique, and a machine learning technique.

3. The method of claim 1, wherein the determination of the current cognitive load score of the person is achieved using at least one machine learning technique.

4. The method of claim 1, wherein the at least one input data associated with a person includes input data associated with at least one of: the person directly, an environment of the person, and an interaction between the person and at least one entity.

5. The method of claim 1, further comprising:
executing the selected at least one predetermined plan.

6. The method of claim 5, wherein at least one resource of an electronic device is utilized for executing the selected at least one predetermined plan.

7. The method of claim 1, wherein the determination of whether a reduction of the current cognitive load score of the person is desirable is achieved using at least one machine learning technique.

8. The method of claim 7, wherein the at least one machine learning technique includes at least one of: a neural network, a recurrent neural network, decision tree learning, a Bayesian network, and clustering.

9. The method of claim 1, wherein determining the current cognitive load score of the person is performed by an artificial intelligence processor.

10. A non-transitory computer readable medium having stored thereon instructions for causing a processing circuitry to perform a process, the process comprising:
determining, based on an analysis of at least one input data associated with a person, a current cognitive load score of the person;
determining, based on the analysis of the at least one input data and the determined current cognitive load score, whether a reduction of the current cognitive load score of the person is desirable; and,
selecting at least one predetermined plan for execution based on when a reduction of the current cognitive load score of the person is desirable.

11. A system for monitoring and managing a cognitive load of a person, comprising:
a processing circuitry; and
a memory, the memory containing instructions that, when executed by the processing circuitry, configure the system to:
determine, based on an analysis of at least one input data associated with a person, a current cognitive load score of the person;
determine, based on the analysis of the at least one input data and the determined current cognitive load score, whether a reduction of the current cognitive load score of the person is desirable; and,
select at least one predetermined plan for execution based on when a reduction of the current cognitive load score of the person is desirable.

12. The system of claim 11, wherein the analysis is of the at least one input data associated with the person is performed using at least one of: a computer vision technique, an audio signal processing technique, and a machine learning technique.

13. The system of claim 11, wherein the determination of the current cognitive load score of the person is achieved using at least one machine learning technique.

14. The system of claim 11, wherein the at least one input data associated with a person includes input data associated with at least one of: the person directly, an environment of the person, and an interaction between the person and at least one entity.

15. The system of claim 11, wherein the system if further configured to:
execute the selected at least one predetermined plan.

16. The system of claim 15, wherein at least one resource of an electronic device is utilized for executing the selected at least one predetermined plan.

17. The system of claim 11, wherein the determination of whether a reduction of the current cognitive load score of the person is desirable is achieved using at least one machine learning technique.

18. The system of claim 17, wherein the at least one machine learning technique includes at least one of: a neural network, a recurrent neural network, decision tree learning, a Bayesian network, and clustering.

19. The system of claim 11, wherein determining the current cognitive load score of the person is performed by an artificial intelligence processor.
